(19) United States
(12) Patent Application Publication
(10) Pub. No.: US 2019/0029877 A1
(43) Pub. Date: Jan. 31, 2019

(54) TRI-MODAL LOCALIZED HEATING GARMENT

(71) Applicant: Max Betkowski, Larkspur, CA (US)

(72) Inventor: Max Betkowski, Larkspur, CA (US)

(21) Appl. No.: 16/151,579

(22) Filed: Oct. 4, 2018

Related U.S. Application Data
(63) Continuation-in-part of application No. 15/474,822, filed on Mar. 30, 2017.

(51) Int. Cl. A61F 7/02; A41D 1/02; A41F 9/00; A61F 7/00; A61N 5/06; A41D 27/20; A41D 1/00

(52) U.S. Cl. CPC A61F 7/02; A41D 2600/10; A41F 9/002; A61F 7/007; A61N 5/0625; A41D 27/205; A41D 1/005; A61F 2007/0086; A41D 2400/12; A61F 2007/023; A61F 2007/0234; A61F 2007/0238; A61N 2005/0645; A61N 2005/0659; A61N 2005/0626; A61N 2005/0666; A61F 2007/008; A61F 2007/0071; A41D 2300/22; A61N 2005/0662; A41D 2400/32; A41D 1/02

(57) ABSTRACT

A garment that heats a portion of a human body during warm-up prior to athletic endeavor having a heater pad member with a size between the size of a common index card and an ordinary sheet of paper for localized penetrating heating of tissue and muscles. Heating is achieved by electrical heating of a heater pad member having a reflective metal foil on one side of the heater pad and thermally conductive PVF sheets on the opposite side. On the one hand, the metal foil reflects optical radiation toward a human body, while on the other hand, the PVF sheets direct infrared radiation into the human body. Control electronics and the heater pad are contained in separate pockets of the garment that are electrically connected together.

### CROSS-REFERENCE TO RELATED APPLICATION

This is a continuation-in-part of U.S. application Ser. No. 15/474,822, filed Mar. 30, 2017.

### TECHNICAL FIELD

The invention relates to body heating and, in particular, to body heating for exercise warm-up and cool down.

### BACKGROUND OF THE INVENTION

In many sports, the body core or limbs attain an activity temperature well above normal body temperature. At the same time, skin pores open to allow more perspiration. It is desirable that the activity temperature be reached gradually to avoid thermal strain on muscles and nerves. To reach the activity temperature, athletes usually perform warm-up exercises, gradually increasing body temperature. Similarly, when sports activity ceases, the body returns to a normal temperature but, to avoid thermal strain on muscles and nerves, as well as to allow gradual closing of skin pores, athletes frequency wear coats or blankets to allow for gradual cool down.

Body heating and cooling devices of various types are known in the prior art. U.S. Pat. Nos. 8,445,819 and 7,115,844 to Ferguson describe a therapeutic heater element for topical application with electrical heating and programmable thermal regulation. As seen in the '819 patent, an adhesive retaining layer is laminated to the back surface of a fabric heater element, which in turn is flush with a wound contact layer. The flexible electric circuit that forms the heater may be a metalized fabric that has been photochemically etched. There is direct skin contact with a heated porous fabric.

U.S. Pat. No. 5,891,189 to Payne, Jr. describes a heating band or belt providing both heat and pressure to sore muscles. The band is an elongate elastomeric member wrapping about the body part to be heated. An electric heating coil member is embedded within the belt. Heat output is controlled by means of a temperature-controlled switch.

U.S. Patent Publication 2016/0213509 of Petitt describes a thermal wrap that further combines electrical nerve stimulation. Thermal media are sandwiched between plastic film and fabric.

U.S. Pat. No. 7,458,106 to Yue describes a glove having a heating element disposed inside of a fabric sleeve. The heating element is apparently less rigid or prone to break and so is both more comfortable for a user and more durable.

U.S. Pat. No. 6,915,641 to Harvie combines the features of both heating and cooling in a system in the form of a vest and belt worn by a user. Although it is electrically operated, the heat (or cooling) is conducted by circulating fluids (liquid or air) in channels through the vest.

U.S. Patent Publication 2014/0277220 of Brennan et al is likewise in the form of a garment worn by a user. It provides selective control over the areas of a patient's body to which heat can be applied. The whole system appears to be battery operated.

U.S. Patent Publication 2015/0290028 of Isserow et al. describes a battery-powered heat applicator for injury sites of a user. It is a multilayer structure with a heat source sandwiched between a power source and a heat-conductive layer and heat application surface.

While all of the heating garments, bands and pads of the prior art are useful, none are specifically designed or adapted to athletic performance pre-activity and post-activity body intermediate level heating. In order to assure that muscles, nerves and skin pores attain an initial desired level of pre-heating, a regulated heating device is needed that is specific for athletic performance. An object of the invention was to devise such a regulated heating device.

### SUMMARY

The above object has been met with a heated garment to be worn by an athlete at warm-up time or cool down time. The garment is a heated wrap that has a length that envelops a human body portion, such as a limb or the human torso, by means of an elastomeric band or tight jacket, allowing the wrap to firmly contact a human body portion, such as an arm or leg or body core, in order to communicate maximum heat, not only by contact but by radiation. The wrap has a heater pad pocket for supporting a heater pad that has a length that is a fraction of the band length, always less than half of the length. To amplify heating, the heater pad has a reflective foil member on a side of the pad away from the human body portion and laminated PVF sheets on the opposite side facing the human body portion for infrared heating of adjacent human tissue and muscles. On the one hand, the reflective foil member transmits optical radiation towards a body portion, while, on the other hand, the laminated PVF sheets transmit infrared radiation from the heater pad a short distance into the body portion, penetrating up to several inches into the body. In this manner heat is transmitted to a body portion in a tri-modal manner, namely direct contact, optical radiation and infrared radiation.

Besides the heater pad pocket, the wrap has at least one pocket containing a battery-powered heating control unit. In a one embodiment, the battery and heating control are integrated as a unit contained in a first pocket, while the heater pad is contained in a second pocket and connected to the control unit. In another version, a separate power pad pocket holds a removable flat battery that communicates power to both the heater pad and the controller in its own pocket. The controller electronics package may include a thermistor and a switch connected between the battery and the heater pad in a normally ON configuration, except that the switch may be turned OFF manually. The controller, using a thermistor or similar device to monitor the heater pad temperature, regulates the heater pad to achieve a predetermined and calibrated maximum temperature. The controller pad pocket may also support an external, user manipulated selector that allows manual selection of one temperature of a series of temperatures in a range of maximum temperatures between 110 degrees F. and 135 degrees F.

Using the selector input, the controller governs the amount of current delivered to the heater pad for achieving the desired maximum temperature.

A garment such as a tightly fitting jacket can support more than one heater pad, with each heater pad associated with a controller pad pocket and a power supply pocket.

### BRIEF DESCRIPTION OF THE DRAWINGS

FIG. 1 is a front plan view of a first embodiment of a garment of the invention.

FIG. 2 is an inverted back plan view of the garment of FIG. 1 with three pockets visible for holding a heater pad, a battery power supply, and a control unit.

FIG. 3 is a sectional plan and electrical diagram of a portion of the garment of FIG. 1.

FIG. 4 is an exploded top view of a heater pad for use with the garment of FIG. 1.

FIG. 5 is a front plan view of a second embodiment of a garment of the invention.

FIG. 6 is a front plan view of a third embodiment of a garment of the invention.

FIG. 7 is an inverted back plan view of the garment of FIG. 6 with two pockets visible for holding a heater pad and a control unit with integrated power supply.

FIG. 8 is a sectional plan and electrical diagram of a portion of the garment of FIG. 6.

### DETAILED DESCRIPTION

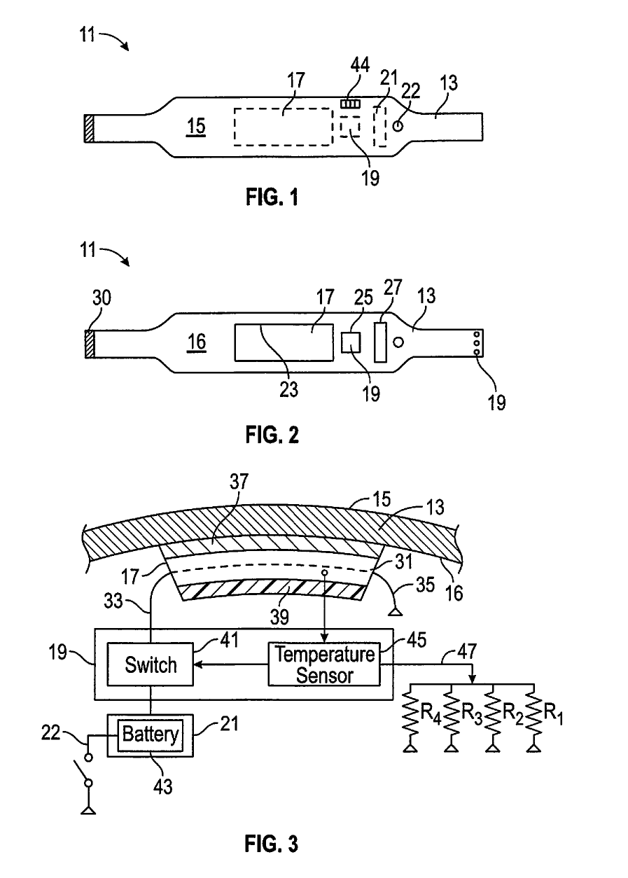

With reference to FIG. 1 a garment 11 is seen to be a wrap member 13 resembling a belt closely fitting around a human torso and a width of approximately 8 inches. It is not necessary for the wrap member to extend about a human torso. Smaller versions of the invention could wrap around a limb, namely an area or leg. A wrap member 13 has an outer surface 15 facing away from a human torso. Within the wrap member are three pockets. A first pocket is a heater pad pocket 17 holding a heater pad. Dashed lines are shown for the pockets because they are not visible from the front of the belt. A second pocket is the controller pocket 19, housing electronic components, which is closely adjacent to the heater pad pocket 15 being separated by about an inch or less. A user temperature selection switch 44, discussed below, is associated with the controller pocket 19. A third pocket is a power pack pocket 21 which is adjacent to the controller pocket. An ON-OFF toggle switch 22, discussed below, is associated with the power pack pocket. The pockets have wires extending between them described below with reference to FIG. 3.

With reference to FIG. 2, the back side of garment 11 shows the outline of pockets 17, 19, and 21 which are within the inner surface 16 of wrap member 13 holding various components of the garment. The heater pad pocket 17 supports a heater pad member that is sandwiched between a metal foil and a plurality of lamented polyvinyl fluoride (PVF) sheets. The foil member in the PVF sheets amplify heat generated in the heater pad member by energy conversion to infrared radiation, directing infrared radiative heat into a human body portion that is adjacent to the heater pad pocket. The pocket has an opening along edge 23 so that the heater pad sandwich can be removed and replaced with a different heater pad sandwich having a different temperature characteristic. A desired temperature for athletic warm ups may be a selected temperature in the range of 110° F.-135° F., for example selected among 110° F., 120° F., 130° F. and 135° F., with a user manipulated switch 22 on the pocket. Electrical connectors between controller electronics in an adjacent controller pocket 19 allow for electrical communication between pockets. The controller pocket 19 is closed along an edge 25 to prevent tampering and to keep control electronics dry, thereby avoiding shorting. A similar construction exists for power pack pocket 21 having a sealed edge 27 protecting a battery that communicates power to the controller pocket 19 which, in turn, communicates power to the heater pad member. The power pack pocket 21 is located a close distance to the control pocket. Each of the controller pocket and power pack pocket has an external, user selectable switch.

Snaps 29 allow wrapping the wrap member 13 around the human torso in a tight-fitting manner. It is intended that the wrap member 13 be made of an elastomeric fabric such as spandex or Lycra, a registered trademark of Invista, both elastic polyurethane fibers, so that the wrap member will be tight fitting when snaps 29 are joined to snap fasteners 31 in a belt manner.

With reference to FIG. 3 the wrap member 13 has an inner surface 16 supporting heater pad pocket 17. Inner surface 16 faces a human torso, as in FIG. 2, while outer surface 15 faces outwardly as in FIG. 1. Heater pad pocket 17 contains a sandwich of three members. The principal member is a heater pad member 31 which has a heater wire, such as a carbon fiber filament or a nichrome wire, with a first end 33 connected to controller pocket 19. A second end 35 of the heater wire is grounded, making a return current path, not shown. Carbon fiber 12-volt heater pads used as automotive seat heaters, sold by Mito Corporation, can be cut to a desired size. Carbon fiber heater pads are described in U.S. Published Appl. No. 2008/0156786 and in U.S. Pat. No. 6,483,087. The carbon fiber heater pad of Appl. No. 2008/0156786 is said to emit radiation in the far infrared which is desirable in the present invention.

On a first major side of the heater pad 31 is a shiny, reflective foil member 37. The foil member may aluminum foil, or preferably a silver foil, having a thickness of only a few thousandth of an inch. The shiny side of the reflective foil member 37 faces the heater pad member 31 for directing optical radiation away from wrap member 13 and towards the human body enclosed by the wrap member.

On the opposite side of the heater pad member is a plurality of laminated PVF (polyvinyl fluoride) sheets 39 whose function is to convert thermally transmitted heat to far infrared radiation which is directed into a human body that the wrap member 13 surrounds. Both the metal foil member 37 and the PVF sheets 39 have approximately the same areawise dimensions of the heater pad member 31 such that the metal foil member, the heater pad member, and the PVF sheets form a sandwich within heater pad pocket 17 that may be laminated together or left as separate members. Typical areawise dimensions for the members is approximately 4 inches in height (width) and 8 inches in length. Regarding area dimensions of the heater pad, preferred width should not exceed 8 inches and preferred length should not exceed 10 inches, i.e. less than the size of a sheet of paper and larger than the size of a common index card. While the wrap member 13 has a thickness of approximately 3 millimeters, the heater pad member has a thickness of less than 2 millimeters. A combination, the wrap member and the heater pad pocket 17 have a combined thickness of approximately 5 millimeters or less as a preferred dimension. This preferred dimension is not essential and other thicknesses could be selected.

Controller pocket 19 contains a normally ON switch 41 receiving wire 33 from the heater pad member 31. The switch 41 supplies electrical current to the heater pad member 31 from a low-voltage battery 43 which is in the closed power pack pocket 21. The battery 43 is preferably a rechargeable battery of the type used in cameras or smart phones. An external user toggle switch 22 can manually turn the battery 43 to an OFF state, although the battery is normally kept ON when the garment is in use.

Within the controller pocket 19 a temperature sensor 43, such as a thermistor measures the temperature in the heater pad member 31 and regulates current into the heater pad to maintain a selected temperature. Other temperature devices, such as thermostats or thermocouples may be substituted with appropriate ancillary support circuits. The heater pad member is built to achieve a maximum temperature, such as in the range of 110° F. to 135° F. The thermistor regulates current into the wire 33 which feeds the serpentine heater wire or carbon fiber wire in the heater member 31. The temperature sensor 45 may be built into the switch 41 and is not necessarily a separate component. In either situation, these electrical members are in the controller pocket 19.

The thermistor temperature sensor 45 allows control of electric current to the heater pad. A logic chip is associated with a user switch that adjusts resistance into the heater pad to achieve a selected temperature. A user manipulated external switch 47, allows selection of an auxiliary resistor R1, R2, R3 or R4 that correspond to one of a selected maximum temperature, for example, 110, 120, 130 or 135° F. set by temperature sensor 45.

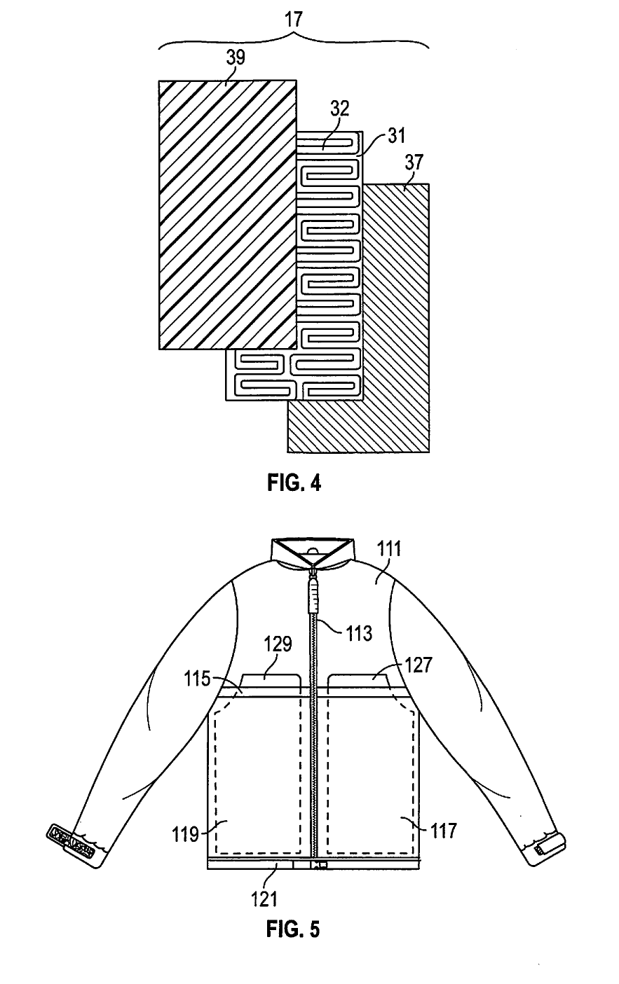

The contents of heater pad pocket 17 are shown in FIG. 4 and include the shiny reflective metal foil member 37, the heater pad member 31 carrying a serpentine pattern of wires 32, sandwiched by the polyvinyl sheets 39 which could be a plurality of thin sheets joined together, with a total thickness of a fraction of one millimeter. The foil member 37, the heater pad member 31 and the polyvinyl sheets 39 may be laminated together.

FIG. 5 shows an alternative garment 111, namely a jacket, that wraps around a human torso and has a slide fastener 113 in front, as well as circumferential belts 115 and 121. Between the belts, a pair heater pad pockets 117 and 119 may be disposed, with each heater pad pocket associated with a corresponding power pack pocket and controller pocket both of which are contained in regions 127 and 129, respectively, adjacent to the heater pad pockets. The heater pad pockets, the power pack pockets and the controller pockets all work as previously described. The jacket embodiment of FIG. 5 can be used not only for athletic warm-ups, but by motorcycle riders and participants in cold weather sports. The garment of the present invention is not restricted to use the torso, or on any particular limb, but may be used on a shoulder, neck or foot, or wherever localized penetrating heat is needed.

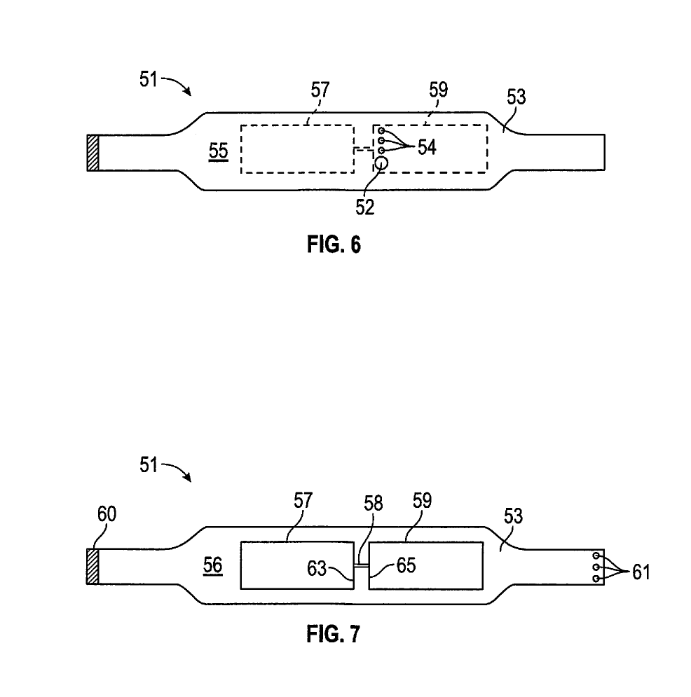

With reference to FIGS. 6 and 7, another embodiment of a garment 51 for localized heating of a human body member according to the present invention features a pocket 59 containing a control electronics package with integrated battery power. As in the previously described embodiments, the garment 51 may resemble a belt, sleeve or alternatively, a jacket. In either case, the garment 51 closely fits around a specified body member to be heated, whether a human torso, shoulder, neck, or limb, and may be made of an elastomeric fabric wrap member 53 with fasteners 60 and 61 that allow it to provide a snug fit against the body member to be heated. An outer surface 55 faces away from the human body, while an inner surface on a backside of the garment 51 is in direct contact with the human body. Also, as in previous embodiments, a heater pad pocket 57, visible only from the backside 56 of the garment 51 when it is turned inside-out, holds a heater pad for directing heat onto the desired body member which is adjacent to the heater pad pocket 57 and its enclosed heater pad. The heater pad is small enough to fit with the pocket 57 in the garment 51, but also has a size that is proportioned to the specific body part to be warmed. The pocket 57 has an opening along edge 63 so that the heater pad can be replaced, as needed (e.g. with one shaped for heating a different body part).

The garment 51 has at least one additional pocket, such as pocket 59 containing control electronics with a flat battery. (There could be still other pockets for one or more additional heating pads associated with other body members to be heated.) The pocket 59 has an opening along edge 65 so that the control electronics package can be removed and replaced, if necessary. The control electronics may be equipped with a USBC-type port both for charging the battery and for transmitting current switch controls for the heating pad element 63.

The exterior of the garment 51 (e.g. on the front surface 55) typically also has an ON-OFF toggle switch 52 and a set of LED indicator lights 54 for temperature and battery charging condition. The ON-OFF button 52 and indicator lights 54 may extend through the garment to the outer surface 55 or at least may be visible through, for example, a transparent window in the garment. The garment material 53 is typically flexible enough to allow the button 52 to be pushed even when it is completely inside the pocket 59. Alternatively (or in addition), a tactile or force-sensitive resistor switch may be woven into the fabric to allow ON/OFF and temperature control functions. Pushing the button one or more times (or through touch-control upon the fabric) may send successive pulses to the controller package to turn the unit ON at a first maximum (130° F.) temperature setting, to lower the temperature setting to a second intermediate (120° F.) temperature, to lower the temperature setting to a third lowest (110° F.) temperature, and finally to turn the unit completely OFF. The indicator lights 54 may illuminate at the distinct temperature settings (130° F. for one light, 120° F. for another light, 110° F. for a third light, and indicate battery charging with a fourth light.

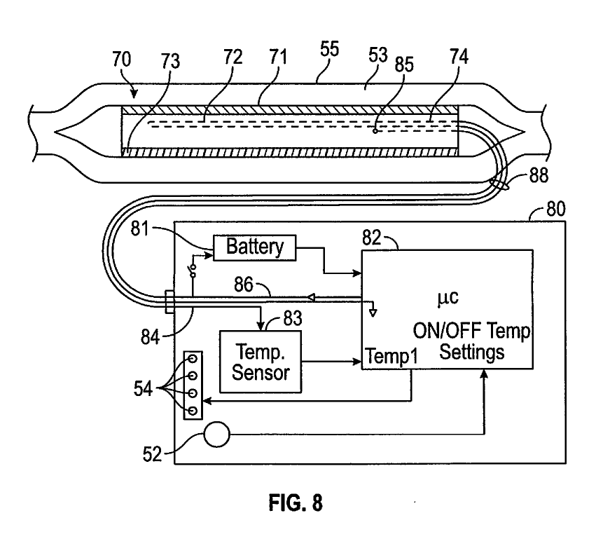

With reference to FIG. 8, as in the first embodiment, the fabric wrap member 53 has an outer surface 55 facing outwardly away from the human body, and an inner surface facing the human body and supporting a heater pad pocket 57 that contains a heater pad 70 in the form of a sandwich of three members 71-73. Like the previously described embodiments, the principle member is a heater pad member 72 which has a heater wire 74 (e.g. carbon fiber filament or nichrome wire) with a first end connected to a battery-powered controller package 80 in a pocket 59. An infrared reflective foil member 71 is on a first major side of the heater pad member 72 for reflecting heat radiation away from the fabric wrap member 53 back towards the human body. On the opposite side of the heater pad member 72 is a lamination of a plurality of polyvinyl fluoride (PVF) sheets 73 of approximately the same areawise dimensions as the heater pad member 72 and whose function is to transmit far infrared thermal radiation from the heater pad member 72 toward the human body.

The pocket 59 contains a battery-powered controller unit 80. The battery 81 (preferably rechargeable) powers a microcontroller 82, a temperature sensor 83 coupled via one wire 83 of the USBC connector 58 to a thermister 85 in the heater pad member 72, and also the heater pad member 72 itself through the USBC connector 58. A user can input a desired temperature setting to the controller unit 80 via the button 52, which is connected to a settings input of the microcontroller 82. As feedback for the controller 82, the temperature sensor 83 supplies a temperature measurement signal from its thermistor 85 to the controller 82 so that it can regulate the current supply that is output on lines 86 to the heater wire 74 and thereby maintain a selected temperature.

Alternatively, the output of controller 82 may increase or decrease a variable resistance state between the battery 81 and the heater wire 84. An ON-OFF toggle switch 52 can turn of the controller 82 and disconnect the battery 61 from the circuit; but additionally, the controller might include a motion sensor or other indicator of whether the garment is being worn, using a timer to automatically shut off the power after some period of inactivity to save battery life. The microcontroller 82 also connects to the indicator lights 54 to turn on one of the lights in accord with the user setting. Finally, the USBC port for the controller unit 80 may be double as a charging input for the battery 81.

What is claimed is:

1. A garment for localized heating of a human body member comprising:
an elastomeric wrap member having opposed outer and inner major surfaces, the wrap member having a length that envelops a human body portion with the outer major surface facing away from the body portion;
a heater pad member supported within a heater pad pocket in the wrap member, having an electrically insulative and thermally conductive fabric confining a heater element with first and second ends, the heater pad member having a lengthwise dimension that is less than half the length of the wrap member;
a shiny, reflective metal foil member within the heater pad pocket of the wrap member with a first foil surface facing heater pad member and a second foil surface facing the inner major surface of the wrap member, with areawise foil member dimensions generally corresponding to areawise dimensions of the heater pad member;
a plurality of laminated PVF sheets forming a infrared radiation transmitting member generally corresponding in areawise dimensions to the heater pad member, the PVF sheets having a combined thickness of less than one millimeter, and having opposed major surfaces with one major surface facing the heater pad, and one major surface arranged for facing a human body member, the foil member and PVF sheets sandwiching the heater pad member therebetween, all within the heater pad pocket;
at least one additional pocket in the wrap member laterally near the flexible heater pad pocket, the at least one additional pocket containing battery-powered control electronics, the battery having a battery electrode electrically communicating power to the control electronics and to a first end of the heater element of the flexible heater pad member through the control electronics; and
a temperature sensor member associated with the controller producing electrical signals governing temperature, the sensor having a temperature set point indicative of a predetermined maximum temperature so that the wrap member maintains a body member near the predetermined maximum temperature;
whereby the reflective foil directs optical radiation from the heater pad member towards a body member and the PVF sheets direct infrared radiation towards the body member, while the heater pad thermally conducts heat towards the body member.

2. The garment of claim 1 wherein the wrap member has a belt shape.

3. The garment of claim 1 wherein the wrap member has a jacket shape.

4. The garment of claim 3 having two heater pad members.

5. The garment of claim 1 wherein the temperature sensor member is a thermistor.

6. The garment of claim 1 wherein the heater element is a carbon fiber heater.

7. The garment of claim 1 wherein the heater element is a serpentine nichrome wire.

8. The garment of claim 1 where the wrap member is made of elastic polyurethane fibers.

9. The garment of claim 1 wherein the heater pad member has a size proportioned to a specific body part to be heated.

10. A garment for localized heating of a human body member comprising:
an elastomeric wrap member having a thickness not exceeding 4 mm and having opposed outer and inner major surfaces, the wrap member having a length that envelops a human body portion with the outer major surface facing away from the body portion;
a heater pad member supported within a heater pad pocket in the wrap member, with a thickness of less than 2 mm and having a non-woven electrically insulative and thermally conductive fabric confining a heater element with first and second ends;
a shiny, reflective metal foil member within the heater pad pocket of the wrap member with a first foil surface facing heater pad member and a second foil surface facing the inner major surface of the wrap member, with areawise foil member dimensions generally corresponding to areawise dimensions of the heater pad member;
a plurality of laminated PVF sheets forming infrared radiation transmitting member generally corresponding in areawise dimensions to the heater pad member, the PVF sheets having a combined thickness of less than one millimeter, and having opposed major surfaces with one major surface facing the heater pad, and one major surface arranged for facing a human body member, the foil member and PVF sheets sandwiching the heater pad member therebetween, all within the heater pad pocket;
at least one additional pocket in the wrap member laterally near the flexible heater pad pocket, the at least one additional pocket containing battery-powered control electronics, the battery having a battery electrode electrically communicating power to the control electronics and to a first end of the heater element of the flexible heater pad member through the control electronics; and
a temperature sensor member associated with the controller producing electrical signals governing temperature, the sensor having a temperature set point indicative of a predetermined maximum temperature so that the wrap member maintains a body member near the predetermined maximum temperature;
whereby the reflective foil directs optical radiation from the heater pad member towards a body member and the PVF sheets direct infrared radiation towards the body member, while the heater pad thermally conducts heat towards the body member.

11. The garment of claim 10 wherein the wrap member has a belt shape.

12. The garment of claim 10 wherein the wrap member has a jacket shape.

13. The garment of claim 12 having two heater pad members.

14. The garment of claim 10 wherein the temperature sensor member is a thermistor.

15. The garment of claim 10 wherein the heater element is a carbon fiber heater.

16. The garment of claim 10 wherein the heater element is a serpentine nichrome wire.

17. The garment of claim 10 where the wrap member is made of elastic polyurethane fibers.

18. The garment of claim 10 wherein the heater pad member has a size proportioned to a specific body part to be heated.
